(19) United States
(12) Patent Application Publication
(10) Pub. No.: US 2026/0041680 A1
(43) Pub. Date: Feb. 12, 2026

(54) METHODS OF TREATING CANCER WITH LONG-ACTING TOPOISOMERASE I INHIBITOR

(71) Applicant: ProLynx LLC, San Francisco, CA (US)

(72) Inventors: Daniel V. SANTI, San Francisco, CA (US); Shaun D. FONTAINE, Concord, CA (US); Gary W. ASHLEY, San Francisco, CA (US)

(73) Assignee: ProLynx LLC, San Francisco, CA (US)

(21) Appl. No.: 19/101,243

(22) PCT Filed: Aug. 3, 2023
(86) PCT No.: PCT/US2023/071580

§ 371 Date: Feb. 4, 2025

Related U.S. Application Data
(60) Provisional application No. 63/370,478, filed on Aug. 4, 2022.

(51) Int. Cl. A61K 31/4745; A61K 9/00; A61K 31/55; A61K 47/60; A61P 35/00

(52) U.S. Cl. CPC A61K 31/4745; A61K 9/0019; A61K 9/0053; A61K 31/55; A61K 47/60; A61P 35/00

(57) ABSTRACT

The disclosure provides method of treating cancer in a patient in need thereof, comprising safely and efficaciously administering to the patient PLX038, a long lasting-PEGylated prodrug of the topoisomerase I inhibitor. The disclosure further provides combination therapies of PLX038 with inhibitors of the DNA damage response (DDR).

### CROSS-REFERENCE TO RELATED APPLICATIONS

This application is a U.S. national stage application under 35 U.S.C. § 371 of International Application No. PCT/US2023/071580 filed Internationally on Aug. 3, 2023 which claims priority to and the benefit of U.S. Provisional Application No. 63/370,478, filed on Aug. 4, 2022, the disclosures of which are incorporated herein by reference in their entirety.

### FIELD

Provided herein are methods and compositions for treating cancer in a patient in need thereof with long-acting Topoisomerase I (Top1) inhibitors, including providing combination therapies of PLX038 with inhibitors of the DNA damage response (DDR).

### BACKGROUND

Topoisomerase I (Top1) inhibitors are a class of chemotherapeutic agents that inhibit DNA replication and induce DNA strand breaks that require poly (ADP-ribose) polymerase (PARP) for their repair. As such, PARP inhibition is highly synergistic in combination with Top1 Inhibitors. In BRCA-wt participants, where PARP inhibitor monotherapy treatment does not demonstrate synthetic lethality, the ability to improve tumor response may be achieved with combination therapy. It is hypothesized that combining PARP inhibitors with Top1 inhibitors will result in increased efficacy in the clinic compared to either agent alone. While the combination of PARP inhibitor and Top1 inhibitor is expected to be synergistic, dose-limiting toxicities have prevented this combination from being dosed at high doses of each drug, thereby limiting its potential clinical utility.

SN-38 is the active metabolite of CPT-11 (irinotecan) that inhibits Top1 and causes DNA strand breakage. As a specific DNA damaging agent, SN-38 enhances cell kill in tumors deficient in the DNA-damage response (DDR; e.g. BRCA1/2-deletions) and when combined with inhibitors of the DNA-damage response (DDR) (e.g. PARP inhibitors). However, synergism is observed in both anti-tumor and toxic effects. Indeed, clinical trials of irinotecan and a PARP inhibitor have shown both activity and toxicities and both drugs required significant dose reductions from their maximum tolerated doses. Notably, in all such studies schedules were such that both DNA damaging agent and were PARP inhibitor concomitantly administered, so maximal systemic exposure of both drugs occurred concurrently and without tumor selectivity.

One approach to achieve selective tumor inhibition involves the use of a “gapped-schedule” to administer the Top1 inhibitor and the PARP inhibitor. Specifically, the tumor-targeted Top1 inhibitor can be administered first, and allowed time (a “gap”) to accumulate in the tumor and clear from the normal tissue. Then, the PARP inhibitor can be introduced to prevent repair of DNA damaged by the Top1 inhibitor. However, the underlying pharmacokinetic profiles of the two agents often precludes the use of a gapped schedule.

PLX038 is long-acting PEGylated prodrug of SN-38. The nanocarrier drug conjugate-designated PLX038 (also known as DFP-13318)—is composed of a 4-arm 40 kDa PEG carrier containing SN-38 moieties attached to the ends of each arm. The prodrug slowly releases SN-38, and has long species-specific elimination tin values of ˜20 hr in mice and ˜5 days in humans. The 4-ARM PEG40 kDa provides a neutral, highly flexible, long half-life nanocarrier with a hydrodynamic diameter of about 15 nm, in accord with ideal properties proposed for nanocarrier tumor accumulation by EPR. Using PLX038, it has been shown that within the tumor environment SN-38 is slowly released over periods of several weeks, substantially increasing tumor exposure to the active drug.

Despite the long half-life and favorable pharmacokinetic profile of PLX038, identifying appropriate dosing schedules remains an important goal. In particular, developing dosing schedules of PLX038 in combination with drugs that target the DNA damage response (DDR) (e.g., PARP inhibitors) that achieve the desired levels of efficacy and safety is an important goal, which has thus far been unmet.

### BRIEF SUMMARY

Provided herein are methods and compositions for treating cancer in a patient in need thereof, comprising administering to the patient a 4-armed PEG that has SN-38 attached by a slowly cleavable releasable linker to the ends of each arm. In one embodiment, the 4-armed PEG/SN-38 is PLX038, which is of the above formula below, where m is 1 and n is approximately 225.

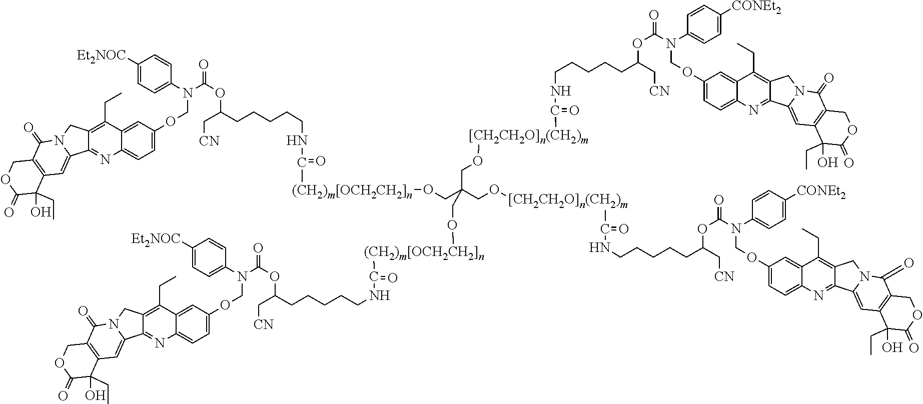

It has been surprisingly discovered that PLX038 can be administered at dose levels that provide high levels of SN-38 exposure in the tumor without providing high levels of SN-38 in the plasma. Accordingly, the safety profile of PLX038 is improved relative to other prodrugs of SN-38 such as irinotecan and EZN-2208. Additionally, the improved therapeutic index enables co-administration with other anti-cancer therapies, particularly inhibitors of the DDR.

In one aspect, the disclosure provides a method of treating cancer in a patient in need thereof, comprising administering a parenteral (e.g., intravenous (IV)) dose of PLX038 once every three weeks, wherein the dose provides a steady state AUC_{(0-∞)} of SN-38 from about 2,000 nM·h to about 8,000 nM·h. These steady state exposures of SN-38 can be achieved without providing plasma concentrations of SN-38 generally associated with toxicity. For instance, in some embodiments, PLX038 can be administered intravenously once every three weeks at a dose that provides a steady state Cₘₐₓ of SN-38 less than 100 nM. In other embodiments, PLX038 can be administered intravenously once every three weeks at a dose that provides a steady state Cₘₐₓ of SN-38 from about 30 nM to about 100 nM.

In some embodiments, the dose of PLX038 administered to the cancer patient once every three weeks is from about 800 mg/m² to about to about 2,000 mg/m². In other embodiments, the dose of PLX038 administered to the cancer patient once every three weeks is from about 1,000 mg/m² to about 2,000 mg/m². In other embodiments, the dose of PLX038 administered to the cancer patient once every three weeks is from about 1,500 mg/m² to about 2,000 mg/m². In other embodiments, the dose of PLX038 administered to the cancer patient once every three weeks is about 1,730 mg/m²

In another aspect, the disclosure provides a method of treating cancer in a patient in need thereof, comprising administering a parenteral (e.g., IV) dose of PLX038 once every three weeks, wherein the dose provides a steady state AUC_{(0-∞)} of SN-38 from about 2,000 nM·h to about 6,000 nM·h, and wherein the PLX038 is administered in combination with an inhibitor of the DDR. In some of the foregoing embodiments, the inhibitor of the DDR is a PARP inhibitor (e.g., rucaparib). These steady state exposures of SN-38 can be achieved without providing plasma concentrations of SN-38 generally associated with toxicity. For instance, in some embodiments, PLX038, when administered in combination with an inhibitor of the DDR, can be administered intravenously once every three weeks at a dose that provides a steady state Cₘₐₓ of less than 50 nM.

In some embodiments, the dose of PLX038 administered once every three weeks, when administered in combination with an inhibitor of the DDR, is from about 350 mg/m² to about 1,300 mg/m². In other embodiments, the dose of PLX038 administered once every three weeks, when administered in combination with an inhibitor of the DDR, is from about 750 mg/m² to about 1,100 mg/m². It will be understood that the PLX038 and the inhibitor of the DDR can be administered simultaneously or sequentially. In preferred embodiments, the inhibitor of the DDR is administered from 2-6 days after the PARP inhibitor in a set dosing schedule.

In particular embodiments, PLX038 is administered in combination with a PARP inhibitor, wherein the PLX038 and the PARP inhibitor are administered at doses as set forth herein. In some such embodiments, the combination of PLX038 and the PARP inhibitor (e.g., rucaparib) is administered to a cancer patient that has a mutation in a gene that normally is helpful in providing a protein that aids in DNA repair. Such tumors are also responsive to topoisomerase inhibitors, such as SN38, since inhibition of topoisomerase causes excess DNA damage that requires DNA repair that is deficient in these tumors. These genes include BRCA1, BRCA2, ATM which encodes ataxia telangiectasia mutated (ATM) kinase and ATR which encodes Rad-3 related (ATR) kinase, among others.

In some embodiments, PLX038 is administered in combination with an inhibitor of the DDR such as an ATR kinase inhibitor, an ATM kinase inhibitor, a CHK1 inhibitor, or a WEE1 inhibitor. In some embodiments, a PARP inhibitor is also administered to the cancer patient.

In some embodiments where PLX038 is administered in combination with a PARP inhibitor (e.g., rucaparib), the PARP inhibitor is administered each cycle only after the PLX038 sufficiently accumulates in the tumor of the cancer patient and is depleted in the plasma of the patient. In one such embodiment, PLX038 is administered by IV infusion on day 1 of every 21-day cycle using a dosing regimen as set forth herein, and the PARP inhibitor (e.g., rucaparib) is administered orally twice daily on days 5-19 of the cycle.

In some embodiments, the PLX038, either alone or in combination with a PARP inhibitor, is administered to a patient that has breast cancer. In some such embodiments, the patient has triple-negative breast cancer. In other embodiments, the patient has ovarian cancer. In other embodiments, the patient has small cell lung cancer.

In some embodiments, the PLX038, either alone or in combination with a PARP inhibitor, is administered to a patient that has a BRCA1 or BRCA2 deficiency. In some embodiments, the patient with the BRCA1 or BRCA2 deficiency has breast cancer. In some such embodiments, the patient with the BRCA1 or BRCA2 deficiency has triple-negative breast cancer. In other embodiments, the patient with the BRCA1 or BRCA2 deficiency has ovarian cancer.

### BRIEF DESCRIPTION OF FIGURES

FIG. 1 shows antitumor activity by PLX038 and irinotecan in HT-29 human colon cancer xenograft model.

FIG. 2 shows antitumor activity by PLX038 and irinotecan in HCT-116 human colon cancer xenograft model

FIG. 3 shows activity by PLX038 and cisplatin in H460 human lung cancer xenograft model.

FIG. 4 shows antitumor activity by PLX038 and irinotecan in NCI-N87 human gastric cancer xenograft model.

FIG. 5 shows antitumor by PLX038 and irinotecan in MKN45 human gastric cancer xenograft model.

FIG. 6 shows antitumor activity by PLX038 and gemcitabine in Panc-1 human pancreatic cancer xenograft model.

FIG. 7 shows antitumor activity by PLX038 and cisplatin in H446 human small cell lung cancer xenograft model.

FIG. 8 shows a plot of the concentration of PLX038 in the serum following administration of a dose of 1,730 mg·m² of PLX038.

FIG. 9 shows a plot of the concentration of SN-38 in the serum following administration of a dose of 1,730 mg·m² of PLX038.

FIG. 10 shows a generalized gapped schedule involving administration of PLX038 and a PARP inhibitor.

FIG. 11 shows complete response in patient with ATM mutated breast adenocarcinoma and multiple prior therapies following treatment with PLX038 and rucaparib.

FIG. 12 shows tumor response in patient with TP53 following treatment with PLX038 and rucaparib.

FIG. 13 shows waterfall-plot displaying best responses among patients enrolled according to dosing schedule 1.

FIG. 14 shows comparison of Steady-State Troughs (Pre-dose) of 300 mg or 400 mg rucaparib given orally twice daily.

### DETAILED DESCRIPTION

Provided herein are methods and compositions for treating cancer in a patient in need thereof, comprising administering to the patient a 4-armed PEG that has SN-38 attached by a slowly cleavable releasable linker to the ends of each arm. In one embodiment, the 4-armed PEG has the following structure:

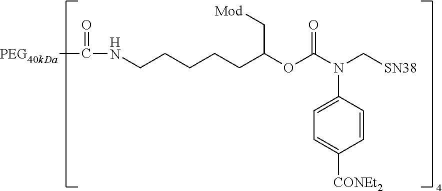

In one embodiment, the 4-armed PEG has the following structure:

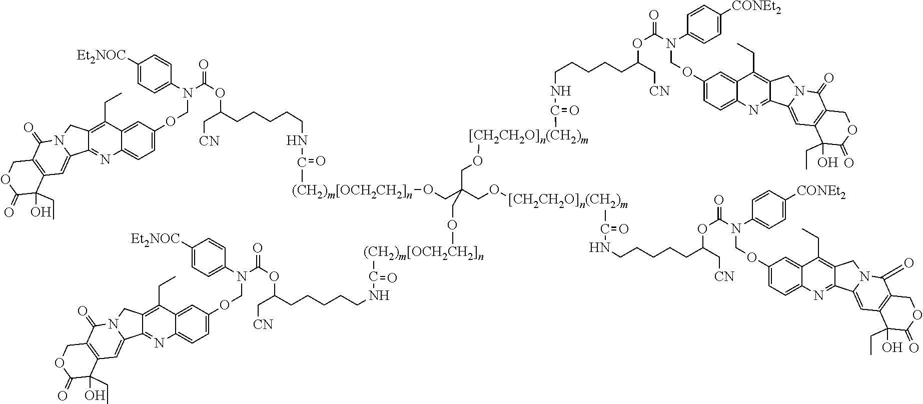

wherein m=1-6 and n is 200-250. In particular embodiments, the conjugate may be PLX038, which is of the above formula where m is 1 and n is approximately 225. PLX038 is disclosed in U.S. Pat. No. 10,016,411, the content of which is incorporated by reference in its entirety. The chemical name for PLX-038 is O-[7-(tetra-polyethylene glycol ether)-carboxamido-1-cyano-2-heptyl]-N-(10′-methoxy-7′-ethyl-camptothecin)-N-[4′-(diethylcarbamoyl)phenyl)] carbamate.

In one aspect, the disclosure provides a method of treating cancer in a patient in need thereof, comprising administering a parenteral (e.g., intravenous (IV)) dose of PLX038 to the patient once every three weeks, wherein the dose provides a steady state AUC (0%) of SN-38 from about 2,000 nM·h to about 8,000 nM·h. In some embodiments, the dose of PLX038 once every three weeks provides a steady state AUC (0-3) of SN-38 from about 3,500 nM·h to about 7,500 nM·h. In some embodiments, the parenteral (e.g., IV) dose of PLX038 once every three weeks provides a steady state AUC_{(0-∞)} of SN-38 from about 4,000 nM·h to about 7,000 nM·h. In some embodiments, the parenteral (e.g.) IV dose of PLX038 once every three weeks provides a steady state AUC_{(0-∞)} of SN-38 from about 4,500 nM·h to about 6,600 nM·h. In some embodiments, the parenteral (e.g., IV) dose of PLX038 once every three weeks provides a steady state AUC_{(0-∞)} of SN-38 from about 5,000 nM·h to about 6,600 nM·h. In some embodiments, the parenteral (e.g., IV) dose of PLX038 once every three weeks provides a steady state AUC_{(0-∞)} of SN-38 from about 5,500 nM·h to about 6,600 nM·h. In some of the foregoing embodiments, PLX038 is administered to a cancer patient that has a genetic defect in a DDR.

The particular steady state exposures of SN-38 set forth above (e.g., about 2,000 nM·h to about 8,000 nM·h, about 3,500 nM·h to about 7,500 nM·h, about 4,000 nM·h to about 7,000 nM·h, about 4,500 n·Mh to 6,600 about nM·h, or about 5,000 nM·h to about 6,600 nM·h) can be achieved without providing plasma concentrations of SN-38 generally associated with toxicity. For instance, in some embodiments, PLX038 can be administered parenterally (e.g., intravenously) once every three weeks at a dose that provides a steady state Cₘₐₓ of SN-38 of less than 100 nM. In other embodiments. PLX038 can be administered intravenously once every three weeks at a dose that provides a steady state Cₘₐₓ of less than 80 nM. In other embodiments. PLX038 can be administered parenterally (e.g., intravenously) once every three weeks at a dose that provides a steady state Cₘₐₓ of SN-38 of less than 70 nM. In other embodiments, PLX038 can be administered parenterally (e.g., intravenously) once every three weeks at a dose that provides a steady state Cₘₐₓ of SN-38 of less than 50 nM. In other embodiments, PLX038 can be administered parenterally (e.g., intravenously) once every three weeks at a dose that provides a steady state Cₘₐₓ of SN-38 of from about 30 nM to about 100 nM. In other embodiments, PLX038 can be administered parenterally (e.g., intravenously) once every three weeks at a dose that provides a steady state Cₘₐₓ of SN-38 of from about 40 nM to about 90 nM. In other embodiments, PLX038 can be administered parenterally (e.g., intravenously) once every three weeks at a dose that provides a steady state Cₘₐₓ of from about 45 nM to about 85 nM. In other embodiments, PLX038 can be administered parenterally (e.g., intravenously) once every three weeks at a dose that provides a steady state Cₘₐₓ of from about 50 nM to about 75 nM. In other embodiments, PLX038 can be administered parenterally (e.g., intravenously) once every three weeks at a dose that provides a steady state Cₘₐₓ of from about 30 nM to about 65 nM. In other embodiments. PLX038 can be administered parenterally (e.g., intravenously) once every three weeks at a dose that provides a steady state Cₘₐₓ of SN-38 of from about 35 nM to about 55 nM. In other embodiments, PLX038 can be administered parenterally (e.g., intravenously) once every three weeks at a dose that provides a steady state Cₘₐₓ of SN-38 of from about 40 nM to about 50 nM. In some of the foregoing embodiments, PLX038 is administered to a cancer patient that has a genetic defect in a DDR.

In some embodiments, the dose of PLX038 once every three weeks is from about 350 mg/m² to about to about 2,000 mg/m². In other embodiments, the dose of PLX038 once every three weeks is from about 1,000 mg/m² to about 2,000 mg/m². In other embodiments, the dose of PLX038 once every three weeks is from about 1,500 mg/m² to about 2,000 mg/m². In other embodiments, the dose of PLX038 once every three weeks is from about 1,500 mg/m² to about 1,800 mg/m². In other embodiments, the dose of PLX038 once every three weeks is from about 1,500 mg/m² to about 1,700 mg/m². In other embodiments, the dose of PLX038 once every three weeks is about 1,000 mg/m². In other embodiments, the dose of PLX038 once every three weeks is about 1,500 mg/m². In other embodiments, the dose of PLX038 once every three weeks is about 1,700 mg/m². In other embodiments, the dose of PLX038 once every three weeks is about 1,730 mg/m². In other embodiments, the dose of PLX038 once every three weeks is about 1,750 mg/m². In other embodiments, the dose of PLX038 once every three weeks is about 1,850 mg/m². In some of the foregoing embodiments, PLX038 is administered to a cancer patient that has a genetic defect in a DDR.

In another aspect, the disclosure provides a method of treating cancer in a patient in need thereof, comprising administering a parenteral (e.g., intravenous) dose of PLX038 once every three weeks, wherein the dose provides a steady state AUC_{(0-∞)} of SN-38 from about 2,000 nM·h to about 6,000 nM·h, and wherein the PLX038 is administered in combination with an inhibitor of the DDR. In some embodiments, the PLX038, when administered in combination with an inhibitor of the DDR, the PLX038 is administered at an IV dose once every three weeks, wherein the dose provides a steady state AUC_{(0-∞)} of SN-38 from about 2,000 nM·h to about 4,500 nM·h. In other embodiments, the PLX038, when administered in combination with an inhibitor of the DDR, is administered at an IV dose once every three weeks, wherein the dose provides a steady state AUC_{(0-∞)} of SN-38 from about 2,500 nM·h to about 4,000 nM·h. In other embodiments, the PLX038, when administered in combination with an inhibitor of the DDR, is administered at an IV dose once every three weeks, wherein the dose provides a steady state AUC_{(0-∞)} of SN-38 from about 2,500 nM·h to about 3,500 nM·h. In other embodiments, the PLX038, when administered in combination with an inhibitor of the DDR, is administered at an IV dose once every three weeks, wherein the dose provides a steady state AUC_{(0-∞)} of SN-38 from about 2,700 nM·h to about 3,700 nM·h.

In some of the foregoing embodiments, the DDR inhibitor is an ATR inhibitor, an ATM inhibitor, a CHK1 inhibitor, or a WEE1 inhibitor. In some of the foregoing embodiments, the inhibitor of DDR is a PARP inhibitor. In some such embodiments, the PARP inhibitor is rucaparib. In other such embodiments, the PARP inhibitor is olaparib. In other such embodiments, the PARP inhibitor is niraparib. In other such embodiments, the PARP inhibitor is talazoparib. In some embodiments, the DDR inhibitor is administered after a period of time in which the PLX038 accumulates in the tumor. For instance, the DDR inhibitor may be administered from two to six days following administration of PLX038. In certain embodiments, the DDR inhibitor is administered 4 days after following administration of PLX038.

The particular steady state exposures of SN-38 set forth above (e.g., about 2,000 nM·h to about 6,000 nM·h, about 2,000 nM·h to about 4,500 nM·h, about 2,500 nM·h to about 4,000 nM·h, about 2,500 nM·h to 3,500 about nM·h, or about 2,700 nM·h to about 3,700 nM·h) when PLX-38 is administered as part of a combination therapy with an inhibitor of the DDR can be achieved without providing plasma concentrations of SN-38 generally associated with toxicity. For instance, in some embodiments, PLX038, when administered in combination with an inhibitor of the DDR, can be administered intravenously once every three weeks at a dose that provides a steady state Cₘₐₓ of less than 50 nM. In other embodiments, PLX038, when administered in combination with an inhibitor of the DDR, can be administered intravenously once every three weeks at a dose that provides a steady state Cₘₐₓ of less than 40 nM. In other embodiments, PLX038, when administered in combination with an inhibitor of the DDR, can be administered intravenously once every three weeks at a dose that provides a steady state Cₘₐₓ of less than 30 nM. In other embodiments. PLX038, when administered in combination with an inhibitor of the DDR, can be administered intravenously once every three weeks at a dose that provides a steady state Cₘₐₓ of less than 20 nM. In other embodiments. PLX038, when administered in combination with an inhibitor of the DDR, can be administered intravenously once every three weeks at a dose that provides a steady state Cₘₐₓ of from about 15 nM to about 50 nM. In other embodiments. PLX038, when administered in combination with an inhibitor of the DDR, can be administered intravenously once every three weeks at a dose that provides a steady state Cₘₐₓ of from about 20 nM to about 40 nM. In other embodiments. PLX038, when administered in combination with an inhibitor of the DDR, can be administered intravenously once every three weeks at a dose that provides a steady state Cₘₐₓ of from about 20 nM to about 30 nM.

In some embodiments, the dose of PLX038 once every three weeks, when administered in combination with an inhibitor of the DDR, is from about 600 mg/m² to about 1,200 mg/m². In other embodiments, the dose of PLX038 once every three weeks, when administered in combination with an inhibitor of the DDR, is from about 750 mg/m² to about 1,100 mg/m². In other embodiments, the dose of PLX038 once every three weeks, when administered in combination with an inhibitor of the DDR, is from about 800 mg/m² to about 1,000 mg/m². In other embodiments, the dose of PLX038 once every three weeks, when administered in combination with an inhibitor of the DDR, is about 800 mg/m². In other embodiments, the dose of PLX038 once every three weeks, when administered in combination with an inhibitor of the DDR, is about 900 mg/m². In other embodiments, the dose of PLX038 once every three weeks, when administered in combination with an inhibitor of the DDR is about 1,000 mg/m². In other embodiments, the dose of PLX038 once every three weeks, when administered in combination with an inhibitor of the DDR is about 1,100 mg/m². In some of the foregoing embodiments, PLX038 is administered to a cancer patient that has a genetic defect in a DDR.

In particular embodiments, PLX038 is administered in combination with a PARP inhibitor, wherein the PLX038 is administered under a dosing regimen as set forth herein. In some such embodiments, the combination of PLX038 and the PARP inhibitor (e.g., rucaparib) is administered to a cancer patient that has a mutation in a gene that normally is helpful in providing a protein that aids in DNA repair takes away this property of the gene. Such tumors are also responsive to topoisomerase inhibitors, such as SN38, since inhibition of topoisomerase causes excess DNA damage that requires DNA repair that is deficient in these tumors. These genes include BRCA1, BRCA2, ATM which encodes ataxia telangiectasia mutated (ATM) kinase and ATR which encodes Rad-3 related (ATR) kinase, among others.

In some embodiments where PLX038 is administered in combination with a PARP inhibitor (e.g., rucaparib), the PARP inhibitor is administered each cycle only after the PLX038 sufficiently accumulates in the tumor of the cancer patient and is depleted in the plasma of the patient. In one such embodiment, PLX038 is administered by IV infusion on day 1 of every 21-day cycle using a dosing regimen as set forth herein, and rucaparib is administered orally twice daily on days 5-19 of the cycle. In some embodiments, the rucaparib is administered at a dose of from about 300 mg to about 600 mg (e.g., about 300 mg, about 400 mg or about 600 mg) twice daily.

In some embodiments, a parenteral (e.g., intravenous) dose of PLX038 of about 1,300 g/m² is administered to the patient on day 1 of every 21 day cycle and an oral dose of about 300 mg of rucaparib is administered orally twice daily to the patient. In other embodiments, a parenteral (e.g., intravenous) dose of about 1,300 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of PLX038 of about 1,300 g/m² is administered to the patient on day 1 of every 21 day cycle and an oral dose of about 600 mg of rucaparib is administered orally twice daily to the patient. In other embodiments, parenteral (e.g., intravenous) dose of about 1,000 g/m² of PLX038 is administered to the patient on day 1 of every 21 day cycle and an oral dose of about 300 mg of rucaparib is administered orally twice daily to the patient. In other embodiments, a parenteral (e.g., intravenous) dose of about 1,000 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of about 1,000 g/m² of PLX038 is administered to the patient on day 1 of every 21 day cycle and an oral dose of about 600 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 300 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 600 mg of rucaparib is administered orally twice daily to the patient.

In other embodiments, PLX038 is administered in combination with both a PARP inhibitor (e.g., rucaparib) and another inhibitor of the DDR such as an ATR inhibitor, an ATM inhibitor, a CHK1 inhibitor, a WEE1 inhibitor.

In some embodiments the cancer to be treated is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer, including small cell carcinoma, cutaneous T cell lymphoma, Hodgkin's lymphoma, non-Hodgkin's lymphoma, cancer of the adrenal cortex, ACTH-producing tumors, non-small cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. In some such embodiments, PLX038 can be administered to a cancer patient that has a genetic defect in a DDR.

In some embodiments, the PLX038, either alone or in combination with a PARP inhibitor, is administered to a patient that has breast cancer. In some such embodiments, the patient has triple-negative breast cancer. In other embodiments, the patient has ovarian cancer. In some such embodiments, the ovarian cancer is platinum-resistant. In other embodiments, the patient has small cell lung cancer. In some such embodiments, PLX038 can be administered to a cancer patient that has a genetic defect in a DDR.

In some embodiments, PLX038 can be administered parenterally to the patient. In some embodiments, the route of administration is intravenous, intra-arterial, intramuscular, or subcutaneous. In a preferred embodiment, PLX038 is administered by intravenous infusion.

### EXAMPLES

### Example 1: HT-29 Colon Cancer (In Vivo)

The antitumor activity and tolerability of PLX038 was studied in the subcutaneous HT-29 human colon cancer xenograft nude rat model. Irinotecan was used as a comparator. PLX038 (50, 100 or 200 mg/kg), irinotecan (70/50 mg/kg) or vehicle (10 mM acetate buffer, pH 5) were administered intravenously once a week for 2 weeks.

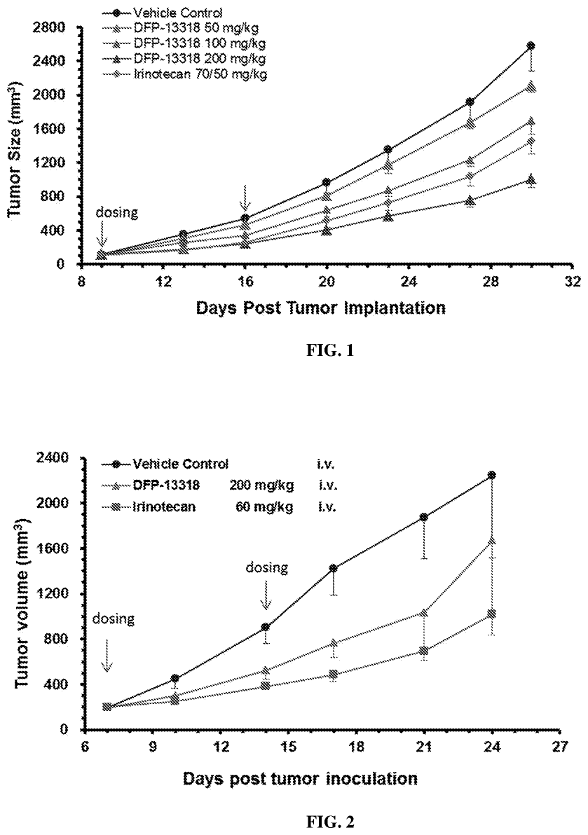

The efficacy of PLX038 and irinotecan at various dose levels are shown in FIG. 1. The tumor growth inhibition (TGI) achieved at PLX038 doses of 50, 100 and 200 mg/kg were 13%, 35% and 61%, respectively. The comparator, irinotecan, achieved 45% TGI. PLX038 caused minimal (2%) body weight loss and no diarrhea. In comparison, irinotecan at 70 mg/kg caused 16% loss in body weight, resulting in the second dose being lowered to 50 mg/kg.

The antitumor activity of PLX038 is attributed to the released SN38, which is less than 4% of total weight from its PEG conjugate. Therefore, PLX038 achieved moderate TGI at a lower molar ratio to SN38/PLX038 compared to irinotecan.

### Example 2: HCT116 Colon Cancer (In Vivo)

The antitumor activity and tolerability of PLX038 was studied in the subcutaneous HCT116 human colon cancer xenograft nude rat model. Irinotecan was used as a comparator. PLX038 (200 mg/kg), irinotecan (60 mg/kg), or vehicle (10 mM acetate buffer, pH 5) were administered intravenously to the rats once a week for 2 weeks.

The efficacy of PLX038 and irinotecan are shown in FIG. 2. PLX038 (200 mg/kg) and irinotecan treatments resulted in 46.6% and 65.4% TGI, respectively. Both treatments were well tolerated.

### Example 3: H460 Lung Cancer (In Vivo)

The in vivo antitumor activity and tolerability of PLX038 was studied in the subcutaneous H460 human lung cancer xenograft model in nude rats. Cisplatin was used as a comparator. PLX038 IV (200 mg/kg), cisplatin intraperitoneal (IP) (5 mg/kg), or vehicle (10 mM acetate buffer, pH 5) were administered to the rats once a week for 2 weeks

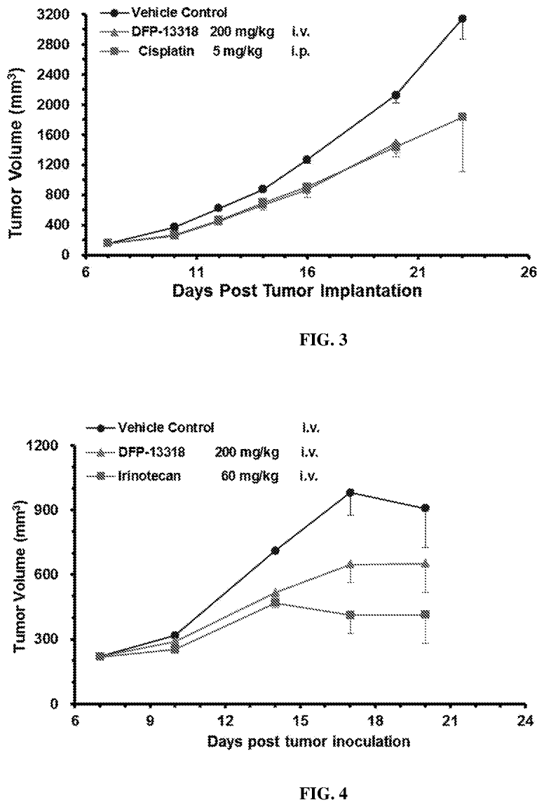

The efficacy of PLX038 and cisplatin are shown in FIG. 3. PLX038 and cisplatin treatments resulted in 30% and 33% TGI, respectively. Both treatments were well-tolerated.

### Example 4: NCI-N87 Gastric Cancer (In Vivo)

The in vivo antitumor activity and tolerability of PLX038 was studied in the subcutaneous NCI-N87 human gastric cancer xenograft model in nude rats. Irinotecan was used as a comparator. PLX038 (200 mg/kg), irinotecan (60 mg/kg), or vehicle (10 mM acetate buffer, pH 5) were administered intravenously to the rats once a week for 2 weeks.

The efficacy of PLX038 and irinotecan are shown in FIG. 4. PLX038 and irinotecan treatments resulted in 34.4% and 57.8% TGI, respectively. Both treatments were well-tolerated.

### Example 5: MKN45 Gastric Cancer (In Vivo)

The in vivo antitumor activity and tolerability of PLX038 was studied in the subcutaneous MKN45 human gastric cancer xenograft model in nude rats. Irinotecan was used as a comparator. PLX038 (200 mg/kg), irinotecan (60 mg/kg), or vehicle (10 mM acetate buffer, pH 5) were administered intravenously to the rats once a week for 2 weeks.

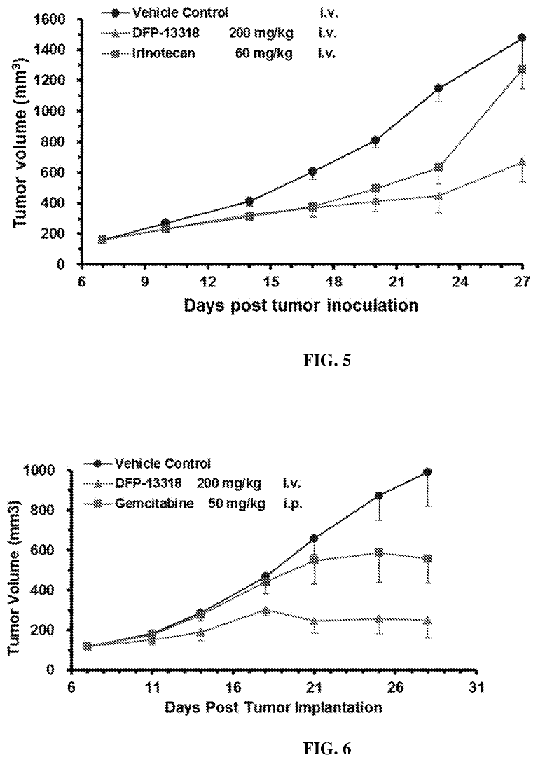

The efficacy of PLX038 and irinotecan are shown in FIG. 5. PLX038 and irinotecan treatments resulted in 59.0% and 45.6% TGI, respectively. Both treatments were well-tolerated.

### Example 6: Panc-1 Pancreatic Cancer (In Vivo)

The in vivo antitumor activity and tolerability of PLX038 was studied in the subcutaneous Panc-1 human pancreatic cancer xenograft model in nude rats. Gemcitabine IP was used as a comparator. PLX038 IV (200 mg/kg), gemcitabine (50 mg/kg), or vehicle (10 mM acetate buffer, pH 5) were administered to the rats once a week for 2 weeks.

The efficacy of PLX038 and gemcitabine are shown in FIG. 6. PLX038 and gemcitabine treatments resulted in 73.1% and 32.5% TGI, respectively. Both treatments were well-tolerated.

### Example 7: H446 Small Cell Lung Cancer (In Vivo)

The in vivo antitumor activity and tolerability of PLX038 IV was studied in the subcutaneous H446 human small cell lung cancer xenograft model in nude rats. Cisplatin IP was used as a comparator. PLX038 (200 mg/kg), cisplatin (5 mg/kg), or vehicle (10 mM acetate buffer, pH 5) were administered to the rats once a week for 2 weeks.

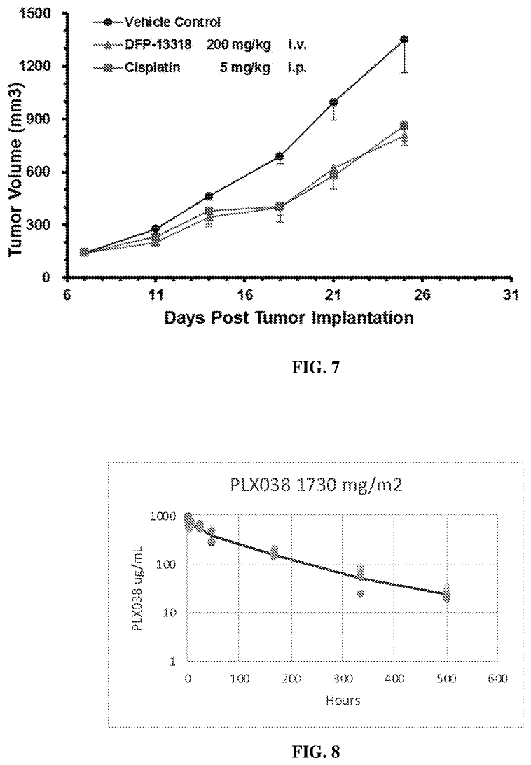

The efficacy of PLX038 and cisplatin are shown in FIG. 7. PLX038 and cisplatin treatments resulted in 41.0% and 36.1% TGI, respectively. Both treatments were well-tolerated.

### Example 8: Phase 1 Dose Escalation of PLX038

The Phase 1 dose escalation trials are being performed in single-dose cohorts. PLX038 was evaluated in 31 participants at dose levels ranging from 115 mg (˜4 mg released SN-38)/m² to 3.6 g (˜23 mg released SN-38)/m² PLX038 administered every 3 weeks. The PLX038 dose of 3060 mg/m² has been identified as the maximal administered dose (MAD) with associated dose limiting toxicities (DLTs) of Grade 4 neutropenia, and Grade 3 nausea, diarrhea, and small intestinal obstruction. Additionally, the intermediate doses of 2800 mg/m² and 2600 mg/m² were not tolerated due to Grade 3 diarrhea in patients treated at these dose levels, and one patient enrolled in the 2000 mg/m² intermediate dose level experienced SARs of Grade 3 dehydration, nausea and vomiting, and Grade 2 diarrhea, all definitely related to therapy.

The median age of patients enrolled is 59.0 years (range: 28-79 years). The majority of patients (33 patients; 84.6%) are white, three patients (7.7%) are Black or African American, one patient (2.6%) is Asian, one patient (2.6%) is of other race, and one patient's (2.6%) race is not reported. At baseline, 4 patients (10.3%) had an Eastern Cooperative Oncology Group (ECOG) performance status of 0, 33 patients (84.6%) had an ECOG performance status of 1, one patient (2.6%) had an ECOG performance status of 0, and one patient's (2.6%) ECOG performance status was unknown.

Thus far, PLX038 has been remarkably safe at doses that provide SN-38 blood levels with area under the curve (AUC) s higher than recommended doses of irinotecan. In addition, because of the low liver exposure of SN-38 from PLX038, the metabolite SN-38G formed from UGT1A1 is low, with plasma SN-38-G/SN-38 ˜0.2. Hence, gastrointestinal (GI) toxicity is neither expected or observed, and UGT1A1 activity is not expected to play a major role in disposition of PLX038; in recognition of the latter, the FDA has allowed full-dose PLX038 treatment of participants heterozygous in UGT1A1*28.

### Example 9: Pharmacokinetic Studies in Humans

Table 1 shows pharmacokinetic (PK) parameters of various administered doses of Q3Wk PLX038 vs 350 mg/m² QWk CPT-11 and two other SN-38 prodrugs, Sacituzumab govitecan and Enz2208. Of note are the following:
Most of the weight of PLX038 is due to the large 40 kDa PEG component; SN-38 content is only 3.5% of the total weight, and considering renal elimination of PLX038, the actual SN-38 present in vivo is only ˜1% of the total weight.
The maximum tolerated dose (MTD) of pegamotecan, a related PEG-camptothecin analog was 3.24 g/m². However, Pegamotecan comprises only 2 molecules of SN38 per PEG, and so the delivered dose of SN28 is only half that from PLX038. Thus, the maximally-tolerated dose of PLX038 (3.06 g/m²) is comparable in terms of total PEG but comprises twice the amount of cytotoxic drug.
At the dose of 2.3 g/m², the delivered free SN-38 is equivalent to the SN-38 formed from the MTD of Q3Wk CPT-11; but, Cmax is ˜50% lower, tin is 10-fold longer, and AUC is over 5-fold higher. The PK benefits of PLX038 vs CPT-11 are favorable.

TABLE 1
Human PK parameters of SN-38 from PLX038 vs other SN-38 prodrugs ^{A}

| | Free SN-38 | SN-38 | SN-38 | SN-38 | SN-38 |
|---|---|---|---|---|---|
| Total SN-38 ^{B} | delivered ^{C} | t_{1/2} | Cₘₐₓ | C_{14 d} ^{D} | AUC ^{E} |
| mg/m²/cycle | mg/m²/cycle | h (n) | nM (n) | nM | nM · h (n) |

PLX038 Q3W

| | | | | | |
|---|---|---|---|---|---|
| 14 | 4 | 97 ± 11.4 (4) | 22 ± 6.2 (6) | 1 | 1,660 ± 560 (4) |
| 21 | 6 | 91 ± 3.7 (2) | 14 ± 4.7 (3) | 2 | 1,756 ± 1071 (2) |
| 31 | 9 | 90 (1) | 24 ± 13 (2) | 3 | 2,513 (1) |
| 46 | 13 | 85 hr | 30 | 2 | 4,000 |
| 62 | 17 | ~100 hr | 46 | 3 | 6,126 |
| 82 | 23 | ~100 hr | 68 | 8 | 11,356 |
| 107 | 32 | 118 ± 31 h | 186 ± 88 | 11.7 ± 1.3 | 17,820 ± 2550 |

CPT-11 Q3Wk ^{F}

| | | | | |
|---|---|---|---|---|
| 227 | 23 | ~12 hr | 143 | 2,000 |

Sacituzumab govitecan ADC (QWk × 2, 3 wk. cycle) [32, 33]

| | | | | | |
|---|---|---|---|---|---|
| 15 | 14 | ~20 hr | 230 | 0.1 | 7,600 |

EZN-2208 (Q3W)

| | | | | | |
|---|---|---|---|---|---|
| 10 | 10 | 30 h | 370 | ~0 | 4,600 |

^{A} PK data of 2,300 mg/m² not yet available; estimates were simulated from lower doses assuming t_{1/2} = 100 h for PLX038 and dose linearity of Cmax.
^{B} SN-38 ~3.5% by weight of PLX038.
^{C} Delivered free SN-38 is ~ ~30% of total SN-38.
^{D} C14 d are estimated from given parameters (below LLOQ).
^{E} AUC0-∞ values are calculated from the models.
^{F} From label.

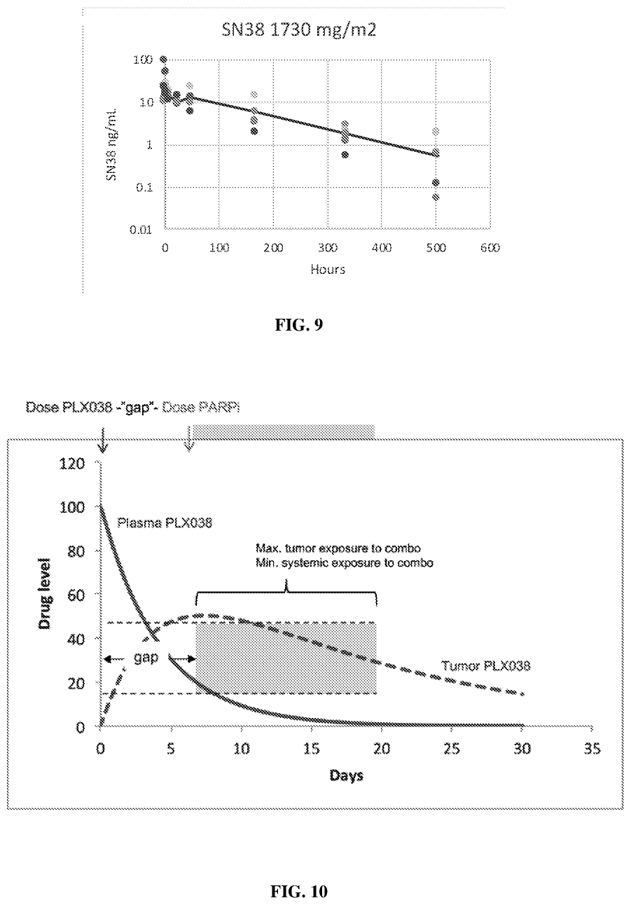

FIG. 8 shows a plot of the concentration of PLX038 in the serum following administration of a dose of 1,730 mg·m² of PLX038. FIG. 9 shows a plot of the concentration of SN-38 in the serum following administration of a dose of 1,730 mg·m² of PLX038. Data are from 3 patients with 2 cycles each.

### Example 10-Combination Therapy (PLX-038/Rucaparib)

Dosing protocols of PLX038 in combination with a PARP inhibitor (rucaparib) were developed to take advantage of the synergy of the combination without concomitant toxicity. The goal is to develop a dosing regimen where the PARP inhibitor is not administered to the cancer patients until efficacious amounts of PLX038 accumulate in the tumor and are excreted from the plasma. A generalized gap schedule in accordance with the disclosure is shown in FIG. 10.

PLX038 is administered as a 1-hour (−10 minutes/+30 minutes) IV infusion on Day 1 of each cycle (21 days). PLX038 vials (220 mg/vial) should be brought to room temperature before use. However, the final diluted product should ideally be used as soon as possible. Vital signs are collected within 1 hour before PLX038 infusions, at least once during the infusions, and within 30 minutes after the completion of the infusion. To calculate the dosage, height measured at screening and weight measured at day 1 of current cycle will be used.

According to schedule 1, rucaparib is given orally at a designated dose twice a day staring 48 hours following the administration of PLX038 on days 3 to 19 of the 21 day cycle. According to schedule 2, rucaparib is given orally at designated dose twice a day on days 5 to 19 of every 21-day cycle. There should be a minimum 5 day window between PLX038 and rucaparib. Rucaparib should be taken at approximately the same times each day. Doses should be taken within 2 hours of the scheduled time.

### Schedule 1

A total of 8 patients were enrolled at dose level 1 [PLX038 1,300 mg/m² (˜50% MTD) and rucaparib 400 mg BID (66% recommended dose)] with a 48-hour window between PLX038 and rucaparib. Six of 8 patients are evaluable for dose limiting toxicity (DLT) assessment. One of six evaluable patients had hematologic DLT of febrile neutropenia, grade 4 thrombocytopenia, and inability to begin subsequent treatment in 21 days due to drug toxicity. Seven patients had non-DLT GI toxicity (most commonly abdominal discomfort, diarrhea, or nausea). In six cases, these symptoms required rucaparib dose reductions or interruptions. In general, these symptoms persisted after rucaparib dose reduction to 300 mg, and to 200 mg in one case.

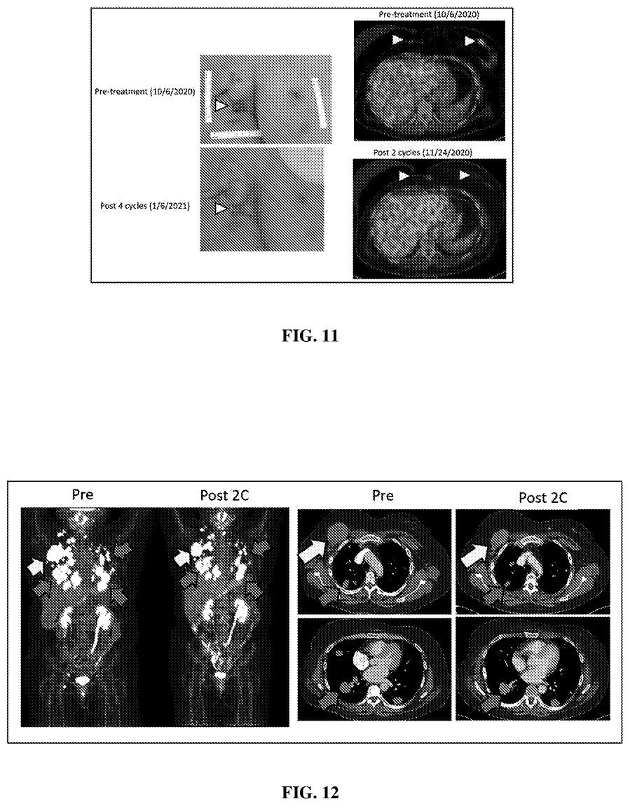

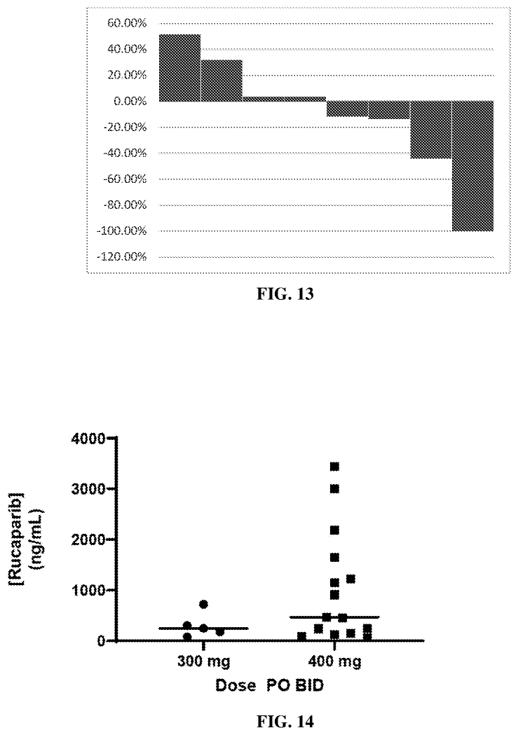

Of seven patients evaluable for efficacy, there were 3 progressive disease (PD), 2 stable disease (SD), 1 partial response (PR), and 1 complete response (CR). Of the two evaluable patients with known tumor homologous recombination deficiencies, there were 1 CR (ATM mutated breast adenocarcinoma, (FIG. 11) and 1 PR (germline TP53 and somatic BRCA1 mutated triple negative breast cancer, FIG. 12), and both patients remained on treatment for 11.5 and 4.5 months respectively. Notably, the responding patients had previously progressed on topoisomerase and PARP inhibitors. Minor responses qualifying us RECIST stable disease were seen in two other patients (FIG. 13).

In summary, data from dose level one demonstrated that, while there are clear signs of efficacy, several patients had intolerable GI toxicities, none of which have met the criteria for DLT. Based on a number of considerations discussed below with respect to Schedule 2, the protocol was amended to decrease the starting dose of rucaparib to 300 mg BID and increase the gap to 5 days between PLX038 and rucaparib.

Samples for PLX038 pharmacokinetic analysis were drawn on Cycle 1 day 1 (C1D1) at the following time points: before PLX038 infusion, end of infusion (EOI) (+5 minutes), 2 (+/−15 minutes) hours, 4 (+/−15 minutes) hours and 24 (+/−30 minutes) hours post EOI, on CID5 just before starting rucaparib, and on C2D1 before the next PLX038 infusion. Samples for rucaparib analysis were collected on C1D7 (optional): before rucaparib, 2 hours (+/−15 minutes), 3 hours (+/−15 minutes), 4 hours (+/−15 minutes) and 24 hours (+/−1 hour) post rucaparib dosing. Samples will also be collected on day 1 of all subsequent cycles before PLX038 dosing.

There were 9 patients enrolled with sufficient PK data for inclusion in this analysis. Sparse sampling of rucaparib precluded a full-time course of concentration data needed to calculate most PK parameters. However, pre-dose (trough) concentrations at steady state (C1D8 and beyond) were analyzed by dose and compared to literature. While no proper statistical comparisons could be made, it appears rucaparib trough concentrations in this study were reasonably consistent with prior data (FIG. 14).

### Schedule 2-Rationale

Data from dosing Schedule 1, discussed above, demonstrates that PLX038 and rucaparib administered at 48-hour gap induces GI adverse events which precludes long-term administration of the combination, despite evidence of anti-tumor activity. The following modifications were considered to the dose escalation schema: a) dose lowering of the rucaparib (currently included in the protocol in schedule 1), and/or b) increase in the time-gap between dosing of PLX038 and the rucaparib. Dose lowering of rucaparib could reduce toxicity, but it may also reduce efficacy; increase in the time-gap between dosing could retain efficacy but reduce toxicity. Nonetheless, as set for the below, good justifications for either approach.

Firstly, dosing schedule 1 may not be optimal for tumor accumulation of PLX038. In rodent models, the tₘₐₓ for tumor accumulation of PLX038 is ˜ 100 hrs. If similar in humans, the 48 hour gap may not be sufficient to allow maximal tumor uptake to take maximum advantage of the highest tumor/systemic PLX038 before BID rucaparib dosing is initiated.

Secondly, the efflux of PLX038 from xenograft tumors is extremely slow compared to normal tissues, so an increased gap time may not affect efficacy (tumor retention of PLX038) but could reduce toxicity (reduced drug in tox target tissue).

Thirdly, pharmacokinetic studies for SN-38 released from PLX038 in the human shows a t_{1/2}=120 h, so increasing the gap from 2 to 5 days should not greatly affect tumor exposure but significantly reduce systemic exposure. The systemic SN-38 at the current gap of 48 hr is 76% of the ˜55 nM Cₘₐₓ, while at a gap of 120 h (5 days) the systemic SN-38 would be decreased to 50% of Cₘₐₓ (or ˜27 nM). While the tumor accumulation of PLX038 will remain unchanged due to the invariant dose and slow exit from the tumor, the systemic level of SN-38 at the time the PARP inhibitor is introduced will be reduced ˜1.5-fold upon changing from a 2 to 5 day gap schedule. This is anticipated to reduce systemic toxicity with less effect on antitumor efficacy.

Fourthly, the current gap may not be sufficient to allow optimal elimination of PLX038 from toxicity target organs (e.g. intestinal epithelial cells) by the time the PARP inhibitor is administered. Unlike the SN-38 formed from CPT-11 which shows high liver/GI-exposure (as evident from high SN-38G/SN-38), the SN-38 from PLX038 has very low liver/GI exposure (as evident from low SN-38G/SN-38); indeed, at the dose of PLX038 used in Schedule 1, SN-38G is barely detectable. Nevertheless, GI exposure to SN-38 released from PLX038 may contribute to the GI toxicity observed with rucaparib. As with SN-38, SN-38G from PLX038 in the human shows a t_{1/2}=120 h. Hence, the GI exposure to SN-38 would be reduced ˜2-fold upon changing from a 2- to 5 day gap schedule which should reduce GI toxicity.

### Schedule 2-Study Design

Up to 3 dose levels of combined treatment of PLX038 and rucaparib will be tested in Phase I (Table 2) with up to 18 subjects enrolled in addition to the 8 participants treated prior to amendment version Mar. 19, 2021. Once an MTD of combined treatment of PLX038 and rucaparib has been determined, up to 35 subjects may be evaluated at MTD in the Phase II cohorts, inclusive of those participants treated at the MTD during the Phase I if they are eligible for the phase II evaluation. Participants will receive treatment in cycles consisting of 21 (+7) days.

TABLE 2
Dosing Schedule

| Dose level | PLX038 every 3 weeks, IV | Rucaparib, PO BID |
|---|---|---|
| Former dose level 1 | 1.3 g/m2 | 400 mg, days 3-19 |
| (completed) | | |
| −1A | 1.0 g/m2 | 300 mg, days 5-19 |
| 1A | 1.3 g/m2 | 300 mg, days 5-19 |
| 2A | 1.3 g/m2 | 400 mg, days 5-19 |
| 3A | 1.3 g/m2 | 600 mg, days 5-19 |

Administration of PLX038 will be every 3 weeks by IV infusion starting on day 1 of cycle 1. The PLX038 dose is 1.3 g/m². Granulocyte-colony stimulating factors (G-CSF) should not be used within at least 24 hours of PLX038 administration. Outside of this restriction window, G-CSF may be administered as clinically indicated. Rucaparib will be administered on days 5 to 19 of a 21-day cycle starting at 300 mg PO BID.

Granulocyte-colony stimulating factors (G-CSF) will be given prophylactically starting on day 2 of each cycle to prevent/treat neutropenia (for both phase 1 and phase 2). G-CSF should not be used within at least 24 hours of PLX038 administration.

Optional biopsies may be performed at: baseline, on day 2 or 3 of Cycle 1 (prior to rucaparib dosing) and between days 7 and 14 of Cycle 1.

The dose of PLX038 and rucaparib may be reduced for toxicity using the following guidelines depending on the toxicities (and drug deemed to be responsible for toxicity) observed.

Based on the below criteria, a maximum of 2 dose reductions will be permitted for each drug (see Table 3 and Table 4). If toxicity persist after 2 dose reductions, participant will be taken off rucaparib or PLX038 treatment.

TABLE 3
Dose reductions for rucaparib

| Dose | Rucaparib dose modifications (mg BID) | | |
|---|---|---|---|
| level | 100% | First dose reduction | Second dose reduction |
| −1A | 300 | 200 | NA (participant will be taken off |
| | | | rucaparib treatment) |
| 1A | 300 | 200 | NA (participant will be taken off |
| | | | rucaparib treatment) |
| 2A | 400 | 300 | 200 |
| 3A | 600 | 400 | 300 |

TABLE 4
Dose reductions for PLX038

| Dose | PLX038 dose modifications (g/m²) | | |
|---|---|---|---|
| level | 100% | First dose reduction | Second dose reduction |
| −1A | 1.0 | 0.8 | 0.6 |
| 2A and 3A | 1.3 | 1.0 | 0.8 |

What is claimed is:

1. A method of treating cancer in a patient in need thereof, comprising administering to the patient a parenteral dose of PLX038 once every three weeks, wherein the dose provides a steady state AUC_{(0-∞)} of SN-38 from about 2.000 nM·h to about 8,000 nM·h.

2. The method of claim 1, wherein said dose provides a steady state AUC_{(0-∞)} of SN-38 from about 3,500 nM·h to about 7,500 nM·h.

3. The method of claim 1, wherein said dose provides a steady state AUC_{(0-∞)} of SN-38 from about 4,000 nM·h to about 7,000 nM·h.

4. The method of claim 1, wherein said dose provides a steady state AUC_{(0-∞)} of SN-38 from about 5,500 nM·h to about 6,400 nM·h.

5. The method of any one of claims 1-4, wherein said dose provides a steady state Cₘₐₓ of SN-38 of less than 100 nM.

6. The method of any one of claims 1-4, wherein said dose provides a steady state Cₘₐₓ of SN-38 of less than 80 nM.

7. The method of any one of claims 1-4, wherein said dose provides a steady state Cₘₐₓ of SN-38 of less than 40 nM.

8. The method of any one of claims 1-4, wherein said dose provides a steady state Cₘₐₓ of SN-38 of from about 30 nM to about 100 nM.

9. The method of any one of claims 1-4, wherein said dose provides a steady state Cₘₐₓ of SN-38 of from about 45 nM to about 85 nM.

10. The method of any one of claims 1-4, wherein said dose provides a steady state Cₘₐₓ of SN-38 of from about 50 nM to about 75 nM.

11. The method of any one of claims 1-10 wherein the dose of PLX038 once every three weeks is from about 350 mg/m² to about to about 2,000 mg/m².

12. The method of any one of claims 1-10, wherein the dose of PLX038 once every three weeks is from about 1,500 mg/m² to about to about 2,000 mg/m².

13. The method of any one of claims 1-10, wherein the dose of PLX038 once every three weeks is from about 1,500 mg/m² to about to about 1,800 mg/m².

14. The method of any one of claims 1-10, wherein the dose of PLX038 once every three weeks is about 1,730 mg/m².

15. The method of any one of claims 1-10, wherein the dose of PLX038 once every three weeks is about 1,000 mg/m².

16. The method of any one of claims 1-10, wherein the dose of PLX038 once every three weeks is about 1,100 mg/m².

17. The method of any one of claims 1-16, wherein the PLX038 is administered in combination with an inhibitor of the DNA damage response (DDR).

18. The method of claim 17, wherein the DDR inhibitor is administered at least two days after the PLX038 is administered.

19. The method of claim 17, wherein the DDR inhibitor is administered four days after the PLX038 is administered.

20. The method of any one of claims 17-19, wherein the DDR inhibitor is a PARP inhibitor.

21. The method of claim 20, wherein the PARP inhibitor is rucaparib.

22. The method of any one of claims 17-19, wherein the DDR inhibitor is a CHK1 inhibitor or a WEE1 inhibitor.

23. The method of any one of claims 17-19, wherein the dose of PLX038 once every three weeks is from about 350 mg/m² to about to about 1,200 mg/m².

24. The method of any one of claims 17-19, wherein the dose of PLX038 once every three weeks is from about 750 mg/m² to about to about 1,100 mg/m².

25. The method of any one of claims 17-19, wherein the dose of PLX038 once every three weeks is from about 800 mg/m² to about to about 1,000 mg/m².

26. The method of any one of claims 17-19, wherein the dose of PLX038 is about 1,000 mg/m².

27. The method of any one of claims 1-26, wherein the cancer patient has a mutation in a gene that provides a protein that aids in DNA repair.

28. The method of claim 27, wherein the gene is a BRCA1 gene.

29. The method of claim 27, wherein the gene is a BRCA2 gene.

30. The method of any one of claims 1-29, where the PLX038 is administered intravenously.

31. A method of treating cancer in a patient in need thereof, comprising administering to the patient a dose of PLX038 and a PARP inhibitor over a 3 week dosing schedule, wherein the PLX038 is administered parenterally on day 1 of the cycle and the PARP inhibitor is administered orally on days 5-19 of the cycle.

32. The method of claim 31, wherein the PLX038 is administered intravenously.

33. The method of claim 31 or claim 32, wherein the PARP inhibitor is administered twice daily.

34. The method of any one of claims 31-33, wherein the PARP inhibitor is rucaparib.

35. The method of any one of claims 31-34, wherein the PLX038 is administered at a dose of 1,300 mg/m².

36. The method of any one of claims 31-34, wherein the PLX038 is administered at a dose of 1,000 mg/m².

37. The method of any one of claims 31-34, wherein the PLX038 is administered at a dose of 850 mg/m².

38. The method of any one of claims 31-34, wherein the PLX038 is administered at a dose that provides a steady state AUC_{(0-∞)} of SN-38 from about 2,500 nM·h to about 8,000 nM·h.

39. The method of any one of claims 31-34, wherein the PLX038 is administered at a dose that provides a steady state AUC_{(0-∞)} of SN-38 from about 4,500 nM·h to about 7,000 nM·h.

40. The method of any one of claims 34-39, wherein the rucaparib is administered twice daily at a dose of 600 mg.

41. The method of any one of claims 34-39, wherein the rucaparib is administered twice daily at a dose of 400 mg.

42. The method of any one of claims 34-39, wherein the rucaparib is administered twice daily at a dose of 300 mg.

43. The method of any one of claims 31-42, wherein the patient has breast cancer.

44. The method of claim 43, wherein the patient has triple-negative breast cancer.

45. The method of any one of claims 31-42, wherein the patient has ovarian cancer.

46. The method of any one of claims 31-42, wherein the patient has small lung cancer.

47. The method of claim 46, wherein the gene is a BRCA1 gene.

48. The method of claim 46, wherein the gene is a BRCA2 gene.
